(19) United States
(12) Patent Application Publication
(10) Pub. No.: US 2018/0195035 A1
(43) Pub. Date: Jul. 12, 2018

(54) Rapid Detection of Human Pathogens in Plant Material Or Water

(71) Applicant: Goddard Labs, Inc., Calverton, NY (US)

(72) Inventor: Noel Goddard, New York, NY (US)

(21) Appl. No.: 15/118,011

(22) PCT Filed: Feb. 12, 2015
(86) PCT No.: PCT/US15/15650

§ 371 Date: Aug. 10, 2016

Related U.S. Application Data
(60) Provisional application No. 61/939,010, filed on Feb. 12, 2014.

(51) Int. Cl. C12M 1/00; C12M 1/26; C12M 1/12; C12M 1/34; C12Q 1/06; C12Q 1/24; C12Q 1/22; G01N 1/10; G01N 1/40; B01D 46/00

(52) U.S. Cl. CPC C12M 47/02; C12M 1/26; C12M 25/16; C12M 1/34; C12Q 1/06; C12Q 1/24; C12Q 1/686; G01N 1/10; G01N 1/405; B01D 46/0004; B01D 46/0028; G01N 2001/4088; C12Q 1/22

(57) ABSTRACT

The present invention provides a composition, a method, and a device for the isolation and detection of human pathogens from a complex liquid mixture. More specifically, the invention provides a resin wherein the particles: (i) are non-magnetic; (ii) are substantially free of cells; (iii) are substantially free of extracellular pathogenic DNA; (iv) are capable of forming reversible complexes with bacteria; and (v) have a minimum average particle diameter of 20 μm and a maximum average particle diameter of 1500 μm. The invention further provides a method for isolating human pathogens from plant material and determining the number of human pathogens in plant material. The invention further provides a method for determining whether the number of human pathogens in plant material exceeds a threshold level of pathogenicity. The invention further provides a device for separating resin particles from an aqueous suspension.

### CROSS-REFERENCE TO RELATED APPLICATIONS

This application is a U.S. National Phase of, and claims priority from, International Application No. PCT/US2015015650, filed 12 Feb. 2015, which claims priority to U.S. Provisional Application No. 61/939,010, filed 12 Feb. 2014, both of which are incorporated herein by reference in their entireties.

### FIELD OF THE INVENTION

The present invention relates generally to the field of detection, identification, and quantitation of contamination of plant material by human pathogens.

### BACKGROUND OF THE INVENTION

Food-borne diseases as well as food spoilage remain a significant burden in the global food supply. The United States Centers for Disease Control and Prevention estimates that each year, foodborne diseases cause illness in 1 in 6 Americans resulting in 128,000 hospitalizations and 3,000 deaths, as well as more than $100 billion in economic loss. The number of multistate outbreaks in the past two years, alone, are an ever present reminder that current food safety practices are insufficient to protect the public.

Accordingly, there is a need to improve microbiological testing for human pathogens in foodstuffs. Microbiological testing is performed with two primary purposes: to establish the absence of human pathogens, and to enumerate total microbial load to verify product quality and shelf-life stability. Both goals are vital to the safety of the global food supply.

However, traditional methods of detecting food borne human pathogens are often time-consuming, laborious, and/or costly.

Because of the low number of pathogenic cells present in food samples, traditional methods of detecting food-borne human pathogens require amplification by growth in culture media. The culturing step is an inexpensive primary diagnostic which offers a qualitative indicator of the presence of a human pathogen when it is not necessary to know the amount of microorganism present in a sample, but only its presence or absence. For a detailed analysis, which in many cases is also required, the culturing step can be followed by isolation, quantitation, biochemical or serological identification, and in some cases, subspecific characterization.

In some instances, it is also desirable to quantify the number of pathogens present in the detection of human pathogens in foodstuffs. Enumeration of pathogens present in a food sample is normally performed by a plate count method. This method involves culturing dilutions of sample suspensions in the interior or on the surface of an agar layer in a Petri dish. Individual pathogens will grow to form individual colonies that can be counted visually. Such colonies are referred to as a colony forming unit (CFU). Counting CFUs is a time consuming endeavor.

The culturing step can take up to several days. Consequently, culturing introduces a significant and potentially critical time delay to the detection of pathogenic contamination. In the case of perishable foods such as produce, the items are often distributed before the microbial testing is completed, resulting in post-distribution recalls.

In an effort to eliminate the culturing step, there has been a reliance on antibody based methods. These methods improve specificity and speed, as well as sensitivity. However, antibody based methodologies are often impractical to use unless very expensive and perishable materials are available in a state of constant readiness. For multiple species identification, multiple antibodies are required. For emergent subspecies, the antibodies may not even exist. Therefore, these methods can be costly and in many aspects is limited in its scope of applicability.

Although antibody based methods can be very sensitive, there are limits on its sensitivity, and, in many cases, cellular amplification methods may still be required.

To overcome the need to amplify the bacteria prior to molecular diagnostic analysis, much effort has been devoted to developing methods of separating and concentrating pathogens from food matrices. Accordingly, it would be advantageous to have a method to detect human pathogens present in a sample in quantities previously undetectable without culturing or cellular amplification. Furthermore, it would be advantageous to be able to quantify the number of pathogens in foodstuffs in a fast, sensitive, and reliable manner.

### BRIEF SUMMARY OF THE INVENTION

In one embodiment, the invention provides a resin wherein the particles: (i) are non-magnetic; (ii) are substantially free of cells (sterilized by ethanol, UV, gamma radiation); (iii) are substantially free of extracellular pathogenic DNA; (iv) are capable of forming reversible complexes with bacteria; and (v) have a minimum average particle diameter of 20 μm and a maximum average particle diameter of 1500 μm.

In another embodiment, the invention provides a reversible complex comprising a human pathogen and a sterilized non-magnetic resin particle as described above.

In another embodiment, the invention provides a method for isolating human pathogens from a liquid.

In another embodiment, the invention provides a method for determining the number of human pathogens in liquid.

In another embodiment, the invention provides a method for determining whether the number of human pathogens in a liquid exceeds a threshold level of pathogenicity.

In another embodiment, the invention provides a device or system for isolating human pathogens from a liquid containing a resin and plant material or water.

### BRIEF DESCRIPTION OF THE DRAWINGS

FIG. 1A-1C depicts an embodiment of the assembled device (1) of the claimed invention, wherein the third container (4) for collecting the flow through is attached.

FIG. 2A-2C depicts an embodiment of the device of the claimed invention shown in FIG. 1, wherein the third container, a sample/eluate collection tube is attached.

FIG. 3 depicts an exploded view of an embodiment of the device of the claimed invention.

FIG. 4A-4B depicts an embodiment of the end cap or lid of the device of the claimed invention. A cross sectional view is also depicted showing the position of the porous barrier (Venting membrane).

FIG. 5A-5B depicts an embodiment of the first container (Sample Collection Container) of the device of the claimed invention.

FIG. 6A-6B depicts an embodiment of adaptor means for connecting the first container (Sample Collection Container) and the second container (Capture Substrate Container) of the claimed invention.

FIG. 7A-7B depicts an embodiment of the second container (Capture Substrate Container) of the device of the claimed invention.

FIG. 8A-8D depicts an embodiment of the second container.

FIG. 9 depicts data derived from capture and release experiments of bacterial DNA and non-bacterial control DNA according to an embodiment of the claimed invention. The figure shows the data for a single capture/release experiment. Each trace is a qPCR reaction quantifying the concentration of genomic DNA. Technical replicates of the qPCR experiments are shown with dashed and solid lines with the same data symbols. Closed circles—Final concentration of E. coli cells captured and released in standard protocol (viability confirmed by plating). Open Circles—Initial concentration of E. coli cells (˜10⁴ cells/mL) processed in protocol. Open triangles—Initial spiked-in concentration of non-E. coli genomic DNA (˜10 ng/mL) processed in protocol. Closed triangles—Final concentration of spiked-in of non-E. coli genomic DNA remaining after standard protocol

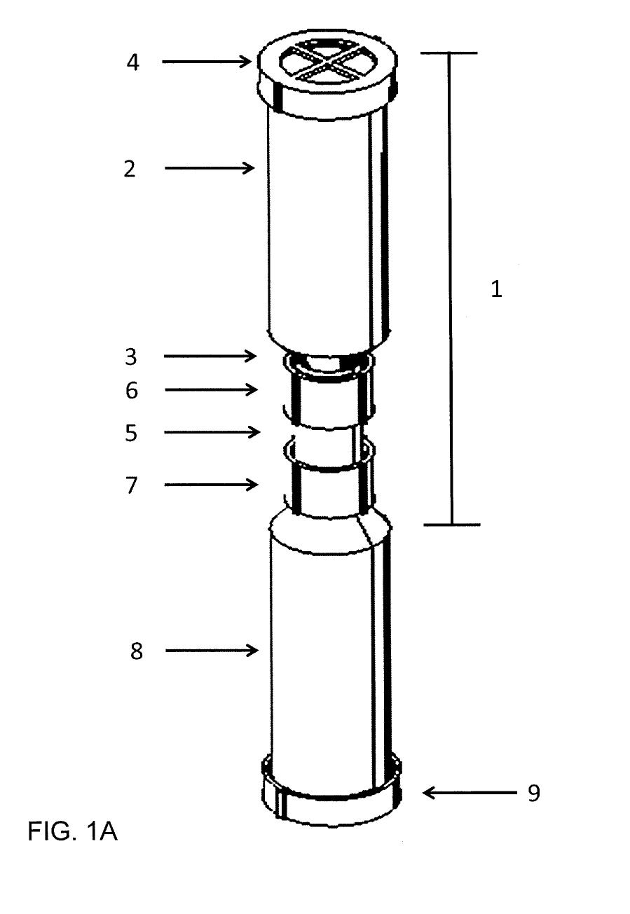

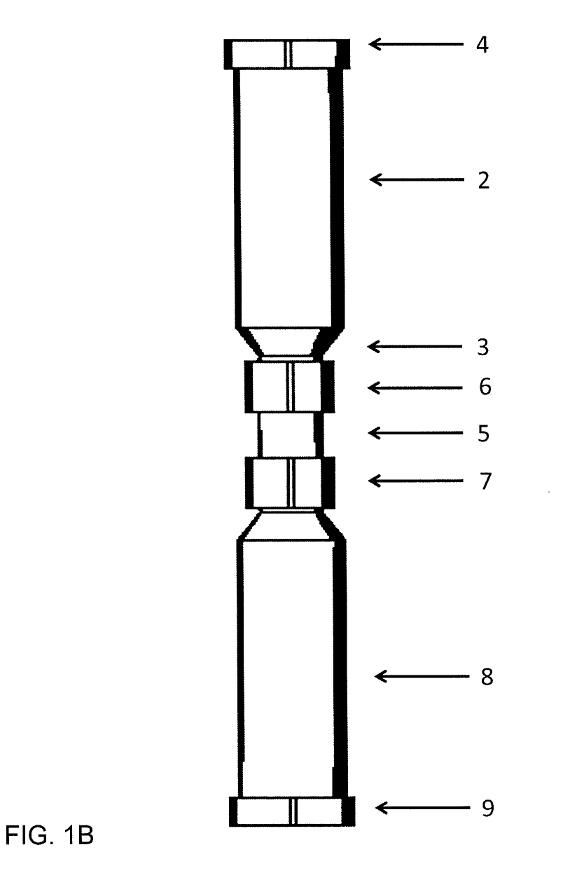

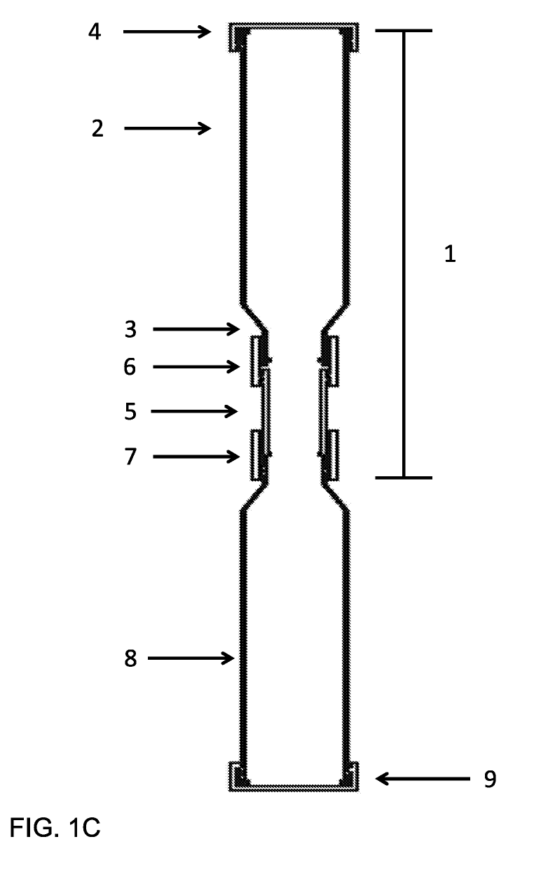

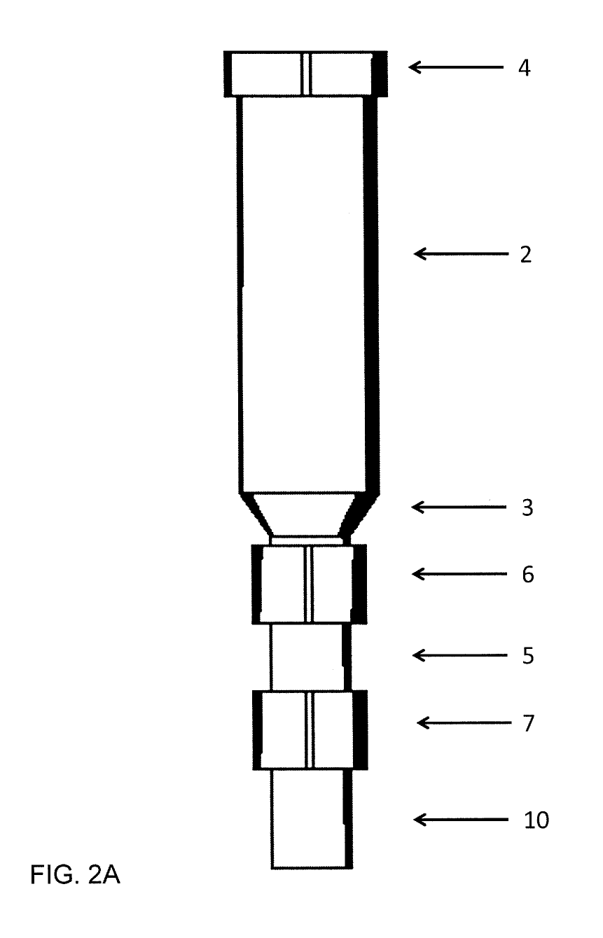

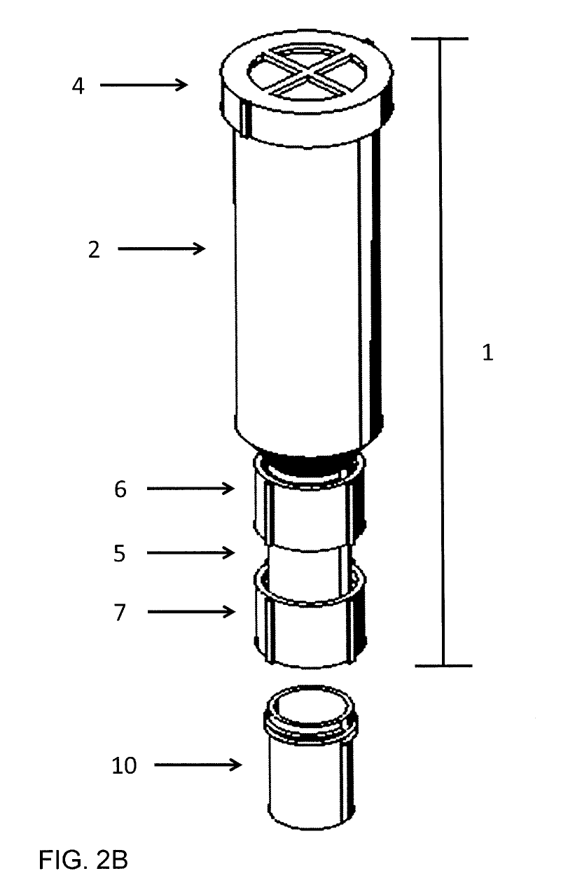

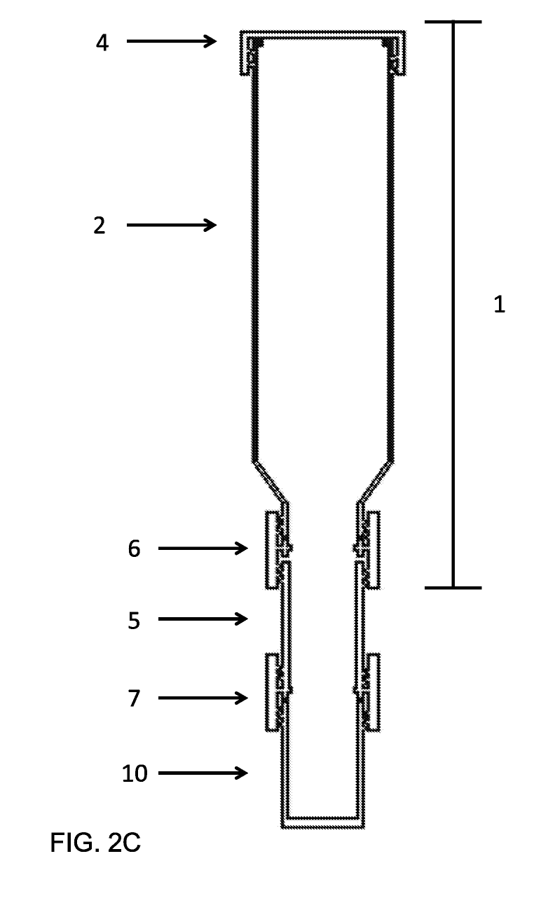

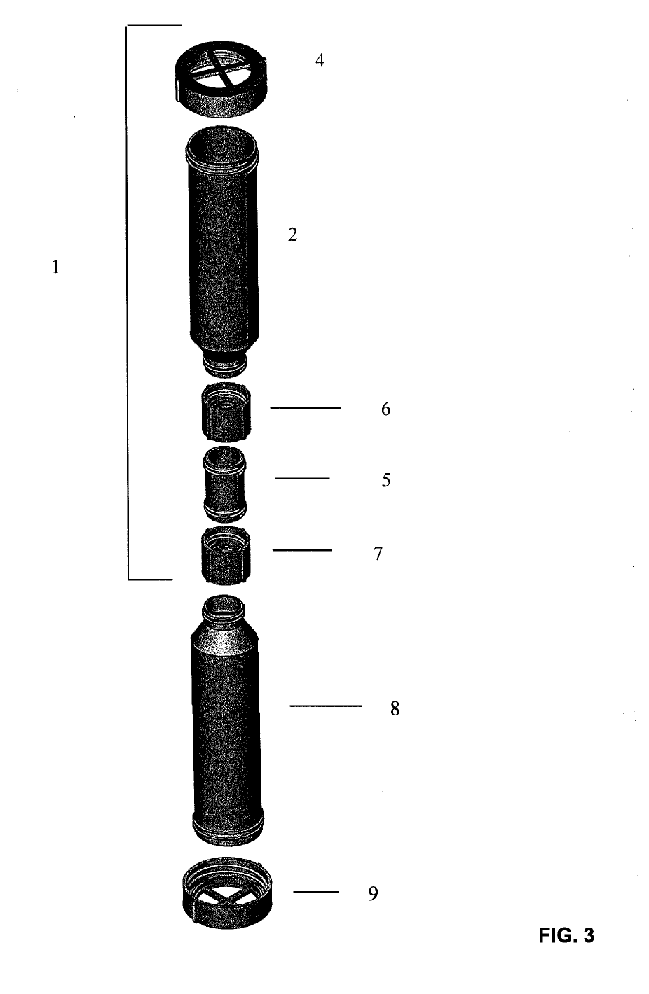

### DETAILED DESCRIPTION OF THE INVENTION

The present invention is generally directed to a composition, a method, and a device for isolating, identifying, and quantifying human pathogens from plant materials, or water.

In one embodiment, the invention provides a composition comprising a basic resin, wherein the particles of the basic resin are unbound non-magnetic; substantially free of cells, and extracellular pathogenic DNA; capable of forming reversible complexes with human pathogens; and have a minimum average particle size of 20 μm and a maximum average particle size of 1500 μm.

A basic resin includes particles that are insoluble in water, and have a positive charge. The charge is conferred to the particles by the presence of molecules or groups on the surface or in interior pores of the particles.

In another embodiment, the invention provides a composition comprising an acidic resin, wherein the particles of the acidic resin are non-magnetic; substantially free of cells, and extracellular pathogenic DNA; capable of forming reversible complexes with human pathogens; and have a minimum average particle size of 20 μm and a maximum average particle size of 1500 μm.

An acidic resin includes particles that are insoluble in water, and have a negative charge. The charge is conferred to the particles by the presence of molecules or groups on the surface or in interior pores of the particles.

The particles possess good mechanical strength, and the material may comprise a natural polymeric substance, a synthetic polymer or co-polymer, or a mixture of natural and synthetics polymers. For example, the polymers are typically carbon, alumina, or silica based. The particles may take the shape of spherical particles, or beads. The particles may be porous or nonporous. This means that the matrix making up the beads may be fully or partially permeable (porous) or completely impermeable to the substance to be removed (non-porous). If the beads are porous, the pores have an average pore size of less than 5 μm in average diameter.

Examples of carbon-based polymers include hydrophilic polymer (e.g. MonoBeads™; Pharmacia, Piscataway, N.J.); cross-linked cellulose (e.g. Sephacel™); cross-linked dextran (e.g. Sephadex™); cross-linked agarose (e.g. Sepharose™); polystyrene, or a co-polymer such as polystyrene-divinylbenzene or one composed of oligoethyleneglycol, glycidylmethacrylate and pentaerythroldimethacrylate, to which are grafted polymerized chains of acrylamide derivatives; Diaion™ Acrylic Gel, Diaion™ Porous; and Diaion™ Highly Porous. The polystyrene may be crosslinked.

In a preferred embodiment, the particles (also known as beads) have a minimum average particle size of 20 μm. In a more preferred embodiment, the minimum average particle size is 100 μm; even more preferably, the minimum average particle size is 200 μm. In another preferred embodiment, the minimum average particle size is 300 μm; even more preferably, the minimum average particle size is 500 μm. In another preferred embodiment, the minimum average particle size is 800 μm; even more preferably, the minimum average particle size is 1000 μm.

In another preferred embodiment, the maximum average particle size is 1500 μm. In a more preferred embodiment, the maximum average particle size is 500 μm; even more preferably, the maximum average particle size is 600 μm. In another preferred embodiment, the maximum average particle size is 800 μm; even more preferably, the maximum average particle size is 1200 μm.

In one embodiment the particles of the resin may be monodisperse. Particles that are monodisperse for the purposes of the present application are those for which the diameter of at least 90% by volume or by weight of the particles varies from the most frequent diameter by not more than 10% of the most frequent diameter.

The particles are non-magnetic. Non-magnetic means, not capable of being magnetized. For example, the non-magnetic particles are not ferromagnetic or ferrimagnetic.

The particles are substantially free of extracellular DNA from pathogenic organisms (pathogenic DNA), and preferably free of any DNA. Extra cellular DNA means DNA found outside the context of the cell. Substantially free from extracellular DNA means, for example, the resin contains picogram quantities or less of DNA per mg of resin. Preferably, a substantially free from DNA product will contain less than about 10 picograms per mg of resin, more preferably, less than about 5 picograms per mg of resin, and most preferably, less than about 1 picograms per mg of resin.

If the particles, which may be purchased or synthesized, contain DNA, methods commonly known in the art may be used to remove the DNA. For example, treatment with ethelene oxide or DNase may be used to remove DNA.

In a preferred embodiment, the particles are substantially free of extracellular RNA from pathogenic organisms (pathogenic RNA), and preferably free of any RNA. Extra cellular RNA means RNA found outside the context of the cell. Substantially free from RNA means, for example, the resin contains picogram quantities or less of RNA per mg of resin. Preferably, a substantially free from RNA product will contain less than about 10 picograms per mg of resin, more preferably, less than about 5 picograms per mg of resin, and most preferably, less than about 1 picograms per mg of resin.

If the particles, which may be purchased or synthesized, contain RNA, methods commonly known in the art may be used to remove the RNA. For example, alkaline hydrolysis or RNase may be used to remove RNA.

The particles are substantially free of cells. As defined herein, cells mean both actively dividing cells, and vegetative cells or spores. Substantially free from cells means that the level of cells in or on the particles are sufficiently low so that assays for determining the levels of specific human pathogens can be accurately performed. Substantially free from cells means, for example, that the maximum number of cells that can be tolerated is preferably no more than 10, more preferably no more than 5, and most preferably no more than 1.

The particles can be sterilized to render them substantially free of cells. Sterilization of the resin particles can be achieved by any known method that does not damage the resin particles. Sterilization in the context of this patent may be roughly defined as a method of killing harmful or unwanted microorganisms through the use of any means having biocidal properties. Examples of such methods include ethylene oxide treatment, ethanol treatment, ultra violet treatment, gamma irradiation, or e-beam sterilization.

The particles are capable of forming reversible complexes with human pathogens. Preferably, human pathogens will bind to the resin particles by way of electrostatic interactions i.e., charge difference.

In one embodiment, the resin includes a polymeric anion exchange resin. Polymeric anion exchange resins are commonly known in the art. The term “polymeric anion exchange resin” includes a positively-charged molecule.

The basic resin may be strongly basic or weakly basic.

In one embodiment, the basic resin includes a strongly basic resin. Strongly basic resins are commonly known in the art. Strongly basic resins are also known as permanently alkaline resins, and dissociate similarly to inorganic bases like NaOH or KOH. Functional groups that comprise strongly basic resins include, for example, quaternary ammonium and phosphonium groups, polyethyleneimine groups, and tertiary sulfonium groups.

In a preferred embodiment, the strongly basic resin includes acryloyl groups. An example of a basic resin comprising acryloyl groups includes poly acrylamido-N-alkyltrimethylammonium groups, for example acrylamido-N-propyltrimethylammonium chloride.

In another preferred embodiment, the strongly basic resin includes quaternary ammonium groups. Examples of quaternary ammonium groups include dimethylethanolammonium, trimethylammonium, triethylammonium, triphenylammonium, trimethylaminoethyl, trimethylaminomethyl, trimethylaminoethyl, diethyl-(2-hydroxypropyl)aminoethyl, and trimethylamino-hydroxypropyl.

Commercially available examples of strongly basic resins include: DIAION™ SA series (Gel Type), DIAION™ PA series (Porous Type), and DIAION™ HPA series (Highly Porous Type). These resins are available from Mitsubishi Chemical Corporation. Other commercially available examples of strongly basic resins include A300, Type II Strong Base Anion Resin, available from Polysciences, Inc. Similar strongly basic resins are available from GE Healthcare Lifesciences Corporation. Other strongly basic resins suitable for use in the claimed invention include A300, which is available from Polysciences, Inc.

In another embodiment, the composition includes a weakly basic resin. Weakly basic anion exchange resins are commonly known in the art, and are referred to as pH dependent resins. Weakly basic resins may comprise primary, secondary, or tertiary amino groups; individually, or in combination.

Some examples of anion exchange resins comprising primary amino groups include polyallyl amine (PAA) based resins, for example, polyallyl amine grafted cellulose. In another embodiment, the primary, secondary, or tertiary amino groups include polyethylene amine. Examples of tertiary amino groups include, for example dimethylaminoethyl (DMAE) and diethylaminoethyl (DEAE). Commercially available examples of DMAE and DEAE resins are available from EMD Millipore. Some examples include Fractogel™ EMD DMAE and Fractogel™ EMD DEAE.

Further commercially available weakly basic resins are available from Mitsubishi Chemical Corporation. Some examples of suitable resins include Diaion™ WA10, WA20, WA21J, and WA30. Similar weakly basic resins are available from GE Healthcare Lifesciences Corporation.

In another embodiment, the resin includes a polymeric cation exchange resin. Polymeric cation exchange resins are commonly known in the art. The term “polymeric cation exchange resin” includes a negatively-charged molecule.

The basic resin may be strongly acidic or weakly acidic.

In another embodiment, the acidic resin includes a strongly acidic resin. Strongly acidic resins are commonly known in the art. Strongly acidic resins are also known as permanently acidic resins, and dissociate similarly to mineral acids like HCl or H₂SO₄. Functional groups that comprise strongly acidic resins include, for example, sulphonic acid, and phosphonic acid groups. Examples of sulphonic groups include sulphopropyl groups. Examples of phosphonic acid groups include alkylphosphonic acid, and aminophosphonic acid.

Commercially available examples of strongly acidic resins are available from Mitsubishi Chemical Corporation. Some examples include DIAION™ SK, UBK, and PK series. Further commercially available weakly basic resins are available from GE Healthcare Lifesciences Corporation. Some examples of suitable resins include SP Sephadex and Source 15S.

In another embodiment, the composition includes a weakly acidic resin. Weakly acidic exchange resins are commonly known in the art. Weakly acidic exchange resins show weak acidity like acetic acid, having the ability to exchange with bases such as NaOH and weak acid salts such as NaHCO₃. Functional groups that comprise weakly acidic resins include carboxylic acid groups and acrylic acid groups. Examples of carboxylic acid groups include carboxymethyl groups. Examples of acrylic acid groups include methacrylic acid groups.

Commercially available examples of weakly acidic resins are available from Mitsubishi Chemical Corporation. Some examples include DIAION™ WK AND WK40 series.

Further commercially available weakly acidic resins are available from GE Healthcare Lifesciences Corporation and EMD Millipore. Some examples of suitable resins include CM Sephadex and Fractogel™ EMD COO. In preferred embodiment, the resin particles as described above are not coated. Not coated means that the resin particles are not coated with any biological material. Coating means attachment by covalent or chemical means. Examples of such biological material include peptides; proteins, such as receptors and monoclonal or polyclonal antibodies; or nucleic acids, such as DNA, RNA, and oligonucleotides.

In a preferred embodiment, the resin particles described above are unbound or are free flowing. Free flowing particles occur, for example, when the particles are in a suspension, or when particles settle out of a suspension sometime after their introduction. The resin particles may, for example, be in an aqueous suspension comprising a homogenate of plant material and human pathogens, or water and human pathogens.

In yet another embodiment, the resin particles described above are not packed in a column. Packed columns are commonly used in chromatography. A packed column is typically formed by a consolidation of a suspension of discrete particles that is pumped, poured, or drawn into a chromatography column. A chromatography column is a container that has a top opening and a bottom opening, and is loaded with particles. Consolidation of the suspension into a packed bed is typically accomplished by filtering it against a particle retaining filter and further compressing the formed filter cake so that it is packed into a volume which is less than the volume that it would have occupied if it had sedimented under the influence of gravity to form a sedimented bed.

In yet another embodiment, the resin particles described above are not bound in a matrix. A matrix is defined as a continuous bed consisting of a single piece of a porous solid material. This is also referred to as a monolith. In a matrix, there are no free-flowing particles in suspension. For example, polyacrylamide-based monolithic beds are made of swollen polyacrylamide gels compressed in a column. Such columns rely on the polymerization of monomers in the chromatographic column. The resulting bed is a rod or plug permeated by channels through which the liquid can pass upon application of pressure or by gravity.

In another embodiment, the resin particles described above are not bound in a membrane or a film. Ion exchange membranes and films are continuous sheets having two surfaces comprising porous solid material to which positively charged ligands are attached or bound.

In another embodiment, the resin particles described above are suspended in water, or aqueous solution.

In another embodiment, the claimed invention provides a method and system of isolating and quantifying human pathogens in plants or water.

Plants include crop plants, in particular monocotyledons such as cereals (wheat, millet, sorghum, rye, triticale, oats, barley, teff, spelt, buckwheat, fonio and quinoa), rice, maize (corn), and/or sugar cane; dicotyledon crops such as beet (such as sugar beet or fodder beet); fruits (such as pomes, stone fruits or soft fruits, for example apples, pears, plums, peaches, almonds, cherries, strawberries, raspberries or blackberries); leguminous plants (such as beans, lentils, peas or soybeans); oil plants (such as rape, mustard, poppy, olives, sunflowers, coconut, castor oil plants, cocoa beans or groundnuts); cucumber plants (such as marrows, cucumbers or melons); fibre plants (such as cotton, flax, hemp or jute); citrus fruit (such as oranges, lemons, grapefruit or mandarins); vegetables (such as spinach, lettuce, cabbages, carrots, tomatoes, potatoes, cucurbits or paprika); lauraceae (such as avocados, cinnamon or camphor); tobacco; nuts; coffee; tea; vines; hops; durian; bananas; natural rubber plants; turfgrasses; and ornamentals (such as flowers, shrubs, broad-leaved trees or evergreens, for example conifers). This list does not represent any limitation.

As defined herein, plant material is defined as any whole plant, portion of a plant, or product of a plant. Plant material includes, without limitation, for example; the stem, seeds, leaves, roots, fruit, or any combination thereof. Further non-limiting examples include various grades of processed fruit and vegetable tissues and fluids, including cut, pureed or liquid plant materials.

Human pathogens can be found on the surface as well as the interior tissue of plants. In order to access human pathogens found in a plant, a plant homogenate must be made. A plant homogenate can be made by any method known in the art. The plant cells are lysed; and pathogens are released and can be separated from the plant tissue.

Methods to lyse plant cells are commonly known in the art. Such methods include grinding, shearing, beating, shocking, sonicating, or a combination thereof. See www.opsdiagnostics.com/notes/ranpri/Homogenization%20Guide%20ver.1.pdf. For example, grinding techniques can be accomplished through manual means such as a mortar and pestle. Automated methods for disrupting tissue can be used as well to lyse cells, and are commonly known in the art. Examples of automated homogenizers include HT Homogenizer from OPS Diagnostics, the Homogenizer from Invitrogen, or Stomacher paddle mixer from Seward Laboratory Systems.

In an embodiment, the minimum amount of plant material to be tested is 1 mg, more preferably 50 mg, even more preferably 100 mg.

In another embodiment, the maximum amount of plant material to be tested is 100 g, more preferably 250 g, even more preferably 1000 g.

In another embodiment, the invention provides a method and system for the isolation and quantification of human pathogens in water or water systems. Examples of suitable water systems include drinking water systems; wastewater facilities; natural water bodies such as streams, rivers, lakes, oceans; storm water collection systems; field and agricultural irrigation systems; greenhouse irrigation systems; groundwater monitoring systems; public swimming beaches and pools; and water used in the processing, manufacturing, or preparation of food.

In another embodiment, the method and system of the claimed invention may be applied to water used for washing food products.

In one embodiment, claimed invention includes testing water going into or coming out of the mentioned water systems by way of sampling the water flow, or by direct access to the water stream.

In an embodiment, the minimum amount of water to be tested is 10 ml, more preferably 50 ml, even more preferably 100 ml.

In another embodiment, the maximum amount of water to be tested is 100 ml, more preferably 250 ml, even more preferably 1000 ml.

In another embodiment, the invention provides a reversible complex comprising a human pathogen and the resin particles described above.

A human pathogen is any microorganism capable of causing disease in a human. In a preferred embodiment, a human pathogen is a bacteria, virus, or fungi that are found in water or plants.

Human pathogens may include a genus of bacteria (e.g., Escherichia, Salmonella, Listeria, streptococci, pseudomonas, Legionella, Cronobacter, Shigella, Vibrio, Campylobacter, and enterococci); distinct variations within a species of bacteria, a serotype (e.g. Typhi, Paratyphi, Enteriditis, Typhimurium and Choleraesuis); a number of related species of bacteria (e.g., coliforms); a strain, genetic variant or subtype of bacteria (e.g. enterohaemorragic, or enterotoxigenic); or an even larger group of bacteria having a common characteristic (e.g., all gram-negative bacteria or all gram-positive bacteria).

In a one embodiment, the complex includes a resin particle described above with a pathogenic gram-negative bacteria. Gram-negative bacteria are bacteria that do not retain crystal violet dye in the Gram staining protocol. Examples of gram-negative bacteria include, without limitation, Escherichia coli (E. coli), Salmonella, Shigella, other Enterobacteriaceae, Pseudomonas, and Legionella

In another preferred embodiment, the complex includes a resin particle described above with a pathogenic gram-positive bacteria. Gram-positive bacteria stain dark blue or violet in the Gram staining protocol. Examples of gram-positive bacteria include, without limitation, Clostridium perfringens (welchii), Clostridium botulinum, and Bacillus cereus.

In certain embodiments, the methods described herein are specific for Listeria, enterohemorrhagic E. coli (EHEC), Salmonella, or Campylobacter.

Further examples of human pathogens include: L. monocytogenes; S. enterica ssp., including but not limited to the serotypes of Typhi, Paratyphi, Enteriditis, Typhimurium and Choleraesuis; C. sakazakii; C. jejuni, C. coli. C. jari; S. dysenteriae, S. flexneri, S. boydii, S. sonnei; V. parahaemolyticus, V. cholerae, and V. vulnificus.

Pathogenic E. coli may be enterohaemorragic (EHEC), enterotoxigenic (ETEC), enteroinvasive (EIEC), and Shiga-like toxin producing (STEC).

In another preferred embodiment, the human pathogen is a virus. Examples of viruses include: hepatitis, and norovirus. Examples of hepatitis include hepatitis A, hepatitis B, hepatitis C, hepatitis D, and hepatitis E. Serotypes, strains, and isolates of norovirus include Norwalk virus, Hawaii virus, Snow Mountain virus, Mexico virus, Desert Shield virus, Southampton virus, Lordsdale virus, and Wilkinson virus.

In another preferred embodiment, the human pathogen is a fungus. Examples include fungi from the genus Aspergillus e.g., A. fumugatus or A. flavus.

In another embodiment, the invention provides a method for isolating human pathogens from plant tissue or water. The method includes preparing an aqueous suspension by contacting a homogenate of plant materials or water comprising human pathogens, and particles comprising a resin as described above, and optionally a first solution. As used herein, contacting includes, for example, mixing or flowing across.

In one embodiment, the aqueous suspension is mixed for a sufficient time to form a complex between the human pathogens and the resin particles; separating the homogenate from the pathogen-particle complex; and separating the human pathogen from the complex with a second solution, thereby obtaining an aqueous mixture comprising the human pathogen sufficiently free of other sources of DNA, or other interfering substances, to permit identification of the human pathogen.

Furthermore, when the human pathogen is a cell, the cell remains intact after separation of the cell from the pathogen-particle complex. In a preferred embodiment, the intact cells are viable. Furthermore, the first and second solutions are sterile and DNA-free. In a preferred embodiment, the first and second solutions are RNA-free. The second solution is compatible with an immunoassay or a qPCR assay; and the pH of the first solution is different from the pH of the second solution.

In an embodiment, the minimum amount of resin is 10 μl, more preferably 50 μl, even more preferably 100 μl.

In another embodiment, the maximum amount of resin is 250 μl, more preferably 500 μl, even more preferably 1000 μl.

The particles described above are mixed with the plant homogenate or water under conditions that provide adequate mixing to distribute the particles evenly throughout the particle plant homogenate or water mixture. Suitable conditions are those that cause turbulence or shearing. Sufficient time for mixing may be readily determined by one skilled in the art, depending on the resin and binding conditions.

Mixing may be accomplished by hand or by use of a laboratory rotator or rocker.

Binding between the resin particles described above and human pathogen may be achieved using a batch treatment, for example, by adding the plant homogenate or water to the resin particles in a vessel, mixing, separating the solid phase (particles), removing the liquid phase (plant homogenate), washing, re-separating, adding the second solution, re-separating, and removing the eluate. The wash step may be performed one or more times to remove any inhibitory material. The complex (of human pathogen and particle as described above) may be washed in any solution that does not disrupt the complex. Suitable solutions include the solution used in complex formation (first solution). In one embodiment, the wash solution includes 20 mM acetic acid at pH 5.

In a preferred embodiment, the resin particles described above are mixed with plant homogenate in sterile water. In another preferred embodiment, the mixture is in a sterile first solution. A solution is defined herein to be a homogeneous mixture of water and optionally buffer, detergent, salt, other additives, and mixtures thereof. Buffers, detergents, and salts are commonly known in the art. Examples of suitable buffers include: TRIS (HCl), sodium citrate or citrate buffer, cacodylic acid, MES, phosphate buffer, MOPS, HEPES, and sodium acetate or acetate buffer. Examples of suitable salts include: NaCl, KCl, and NH₄Cl. Examples of suitable detergents include: SDS, Tween, CHAPS, and Triton.

To prevent non-specific binding and enhance capture of human pathogens the use of non-specific blocking agents has been contemplated. These agents may be added to the initial sample or with the first or wash solution. Examples of suitable agents include cysteine, imidazole, polyvinylpyrrolidone, dextran sulfate, or other non-protein blocking agents. The agent may be present in an amount sufficient to promote binding of the human pathogen and prevent non-specific binding.

Any solution that allows complex formation between the human pathogen and the particles as described above may be used. A person of ordinary skill in the art would understand that the relative amounts of the solution components can be varied, and still allow for complex formation between the human pathogen and resin particles.

Any solution that disrupts the complex between the human pathogen and the particles as described above may be used. A person of ordinary skill in the art would understand that the relative amounts of the solution components can be varied, and still allow for disruption of the complex between human pathogen and resin particles.

When the particle includes a weakly basic resin, the first solution has a minimum pH of about 5 and a maximum pH of about 9. The second solution has a minimum pH of about 3 and a maximum pH of about 6.

When the particle includes a strongly basic resin, the first solution has a minimum pH of about 5 and a maximum pH of about 9. The second solution has a minimum pH of about 3 and a maximum pH of about 6.

In another preferred embodiment, the first solution includes 5 mM cysteine and 20 mM acetic acid having a pH of about 5.

In another preferred embodiment, the second solution includes 1M acetic acid having a pH of about 3.

When the particle includes a weakly acidic resin, the first solution has a minimum pH of about 7 and a maximum pH of about 9. The second solution has a minimum pH of about 3 and a maximum pH of about 6.

When the particle includes a strongly acidic resin, the first solution has a minimum pH of about 7 and a maximum pH of about 9. The second solution has a minimum pH of about 3 and a maximum pH of about 6.

In another preferred embodiment, the first solution includes 0.1 M TRIS HCl, having a pH of about 8.5

In another preferred embodiment, the second solution includes 0.1 M acetic acid having a pH of about 4.

A person of ordinary skill in the art would understand that the relative amounts of the components of the second solution can be varied, and still allow for elution of the human pathogens.

Electrostatic based complex formation between human pathogens and the resin particles described above is dependent upon the surface charge of the resin particle as well as of the human pathogens. The surface charge is influenced by pH and ionic strength of the suspending medium.

Since direct measurement of surface charge of human pathogens is difficult, these interactions are often characterized by the zeta potential. Bacterial zeta potentials can be deduced from electrophoretic mobility measurements, as is known in the art. Accordingly, selective binding and elution of different human pathogens, as well as viable bacteria can be accomplished based upon differences of zeta potential, and adjusting the components or characteristics of the elution solution.

The eluted cells are sufficiently free of DNA and/or RNA from sources other than human pathogens to permit analysis of the plant material.

In a preferred embodiment the purity of the eluted human pathogen is greater than 95%, 90%, 85%, 80%, 75%, 70%, 65%, 60%, 55%, 50%, 45%, 40%, 35%, 30%, 25%, and 20% by weight.

In another embodiment, the eluted cells may be analyzed by an immunological based assay. An example of a suitable assay is Enzyme Linked Immunosorbent Assay (ELISA).

In another embodiment of the invention, the eluted human pathogens is counted. Counting of the bacteria can be done by any known method, for example, by qPCR, or flow cytometry. Manual methods include use of a haemocytometer or CFU plating assay. Automated cell counters such as a TC-10 automated cell counter from Bio-Rad may also be used.

In another embodiment, the invention provides a method for determining whether the number of human pathogens in the plant tissue exceeds a threshold level of pathogenicity.

A threshold level depends, in large part, upon the sample being tested, the type of microorganism suspected of being present in the sample, and any industry- or government-imposed standards related to maximum microorganism concentration. The determination of an appropriate threshold level for a particular sample to be tested may readily be determined by the skilled artisan based upon these and any other criteria established for a suitable application.

In one embodiment, a threshold level is the maximum level considered acceptable by government or industry regulatory standards. In certain embodiments, the threshold level is at least 1×10⁻² cells/g, 5×10⁻² cells/g, 1×10⁻¹ cells/g, 5×10⁻ cells/g, 1×10¹ cells/g, 5×10¹ cells/g, 1×10² cells/g, 5×10² cells/g, 1×10³ cells/g, 5×10³ cells/g, 1×10⁴ cells/g, 5×10⁴ cells/g, 1×10⁵ cells/g, 5×10⁵ cells/g, 1×10⁶ cells/g, or 5×10⁶ cells/g, or any integer value falling between these levels. The units represent pathogenic cells per gram of plant material. In another embodiment, these levels may be of colony forming units (CFU) per gram of plant material, depending upon the quantitation method being used. In yet another preferred embodiment, the threshold is 10 CFU per 25 g of plant matter.

A threshold level may also be determined based upon the presence of an increased amount of a human pathogen or, pathogenic material, in sample as compared to a control sample. In certain embodiments, the presence of human pathogens, or human pathogenic material, is detected if the sample contains at least two-, at least three-, at least four-, at least five- or at least ten-fold more human pathogens than a control sample. Depending upon the nature of detection being performed, in certain embodiments, a control sample may be a sample known to not contain the human pathogens, or pathogenic material, being detected (e.g., a buffer solution), or it may be a sample containing a pre-determined or acceptable amount of a human pathogen, or pathogenic material.

The pathogenic material includes any component of a human pathogen that may be detected or quantified. Non-limiting examples include DNA and RNA. Quantitation of DNA or RNA can be accomplished by any known method. Examples include qPCR, microarray, or any hybridization based assay.

In another embodiment, the invention provides a device for isolating human pathogens from plant material or liquid. The device (1) includes at least two releasably coupled containers, a first container (2) and second container (5); each container having a chamber with two openings. Each container has a length and a width, wherein the length is defined by the distance between the two openings. No particular limitation is placed on the material of the body. Materials that can be used for the container include synthetic resins and glasses.

The first container (2) optionally includes an internal conical skirt portion (3) formed adjacent the outlet port for facilitating the flow of the liquid through the outlet port. Specifically, the skirt portion (3) defines a funnel region of the internal main chamber of the first container (2) for diverting the flow of the liquid under the influence of gravity or pressure.

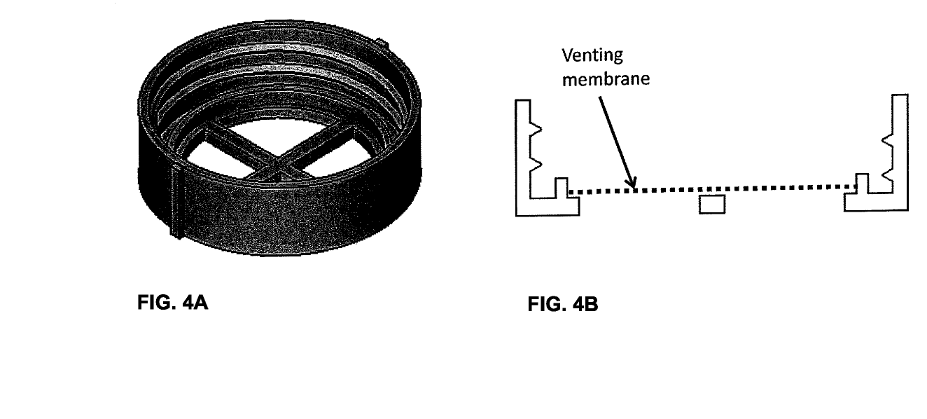

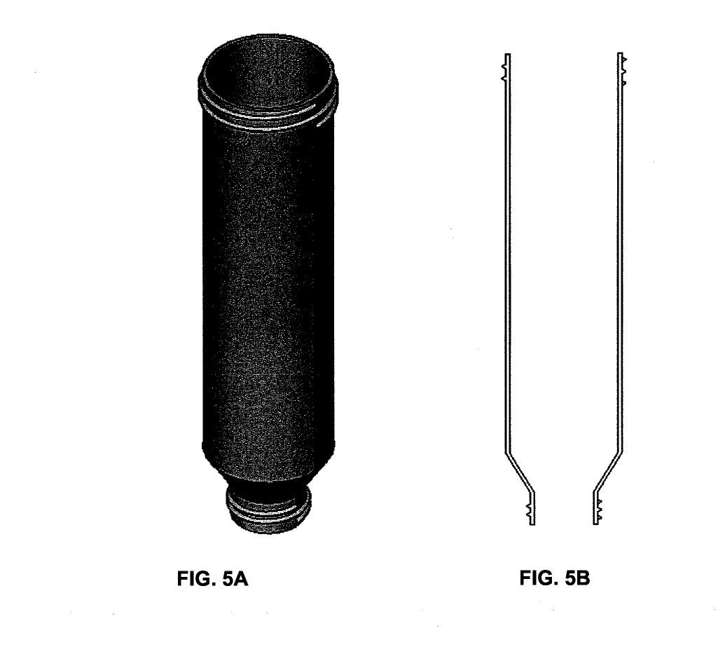

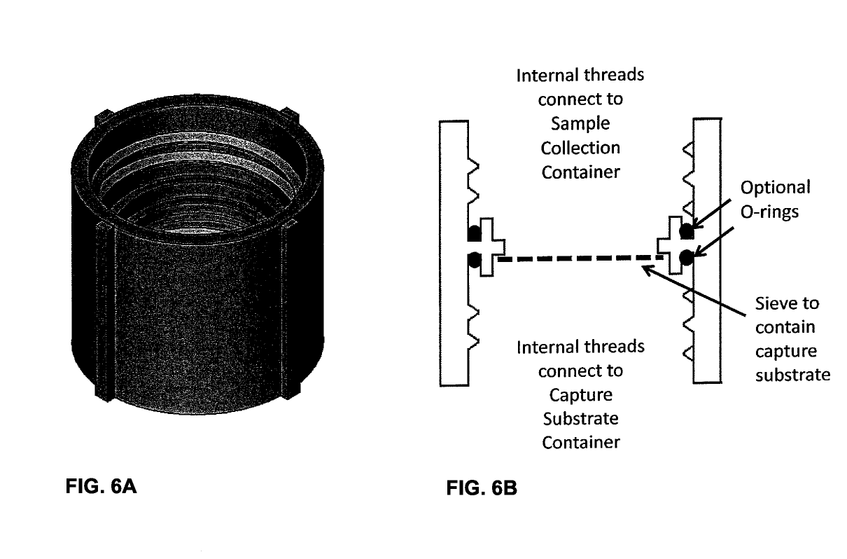

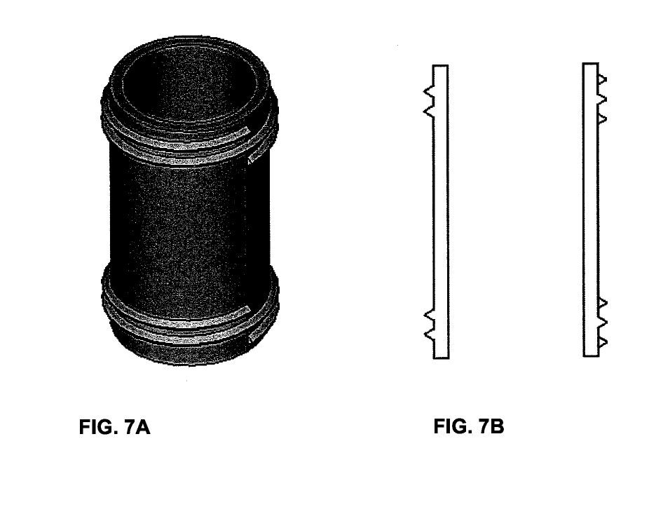

The first container has a cap (4) that can be fastened to the open end of the container by any known fastening means. The cap may comprise a porous barrier (see FIG. 4) to prevent the entry of bacteria or particulate matter, and maintain sterility within the chamber. Furthermore, the barrier may be liquid impermeable, but gas permeable An example of a suitable porous barrier includes Emflon and Supor R membrane, available from Pall Corporation.

The first container (2) may optionally have a port allowing for attachment of a pump or syringe to provide air or create positive pressure. In one embodiment, the first container (2) further comprises a port, which may be located on a side wall of said first container. The port may comprise a porous barrier as described above. The addition of air or creation of positive pressure will facilitate liquid flow out of the first container (2) into the second container (5). For example, the port may include a pressure activated luer lock valve. In another example, the port may include a luer lock with a vented cap.

In one embodiment, the first container includes a plunger or other mechanical means to provide force or positive pressure to assist the flow of liquid out of the first container. For example, the container includes a plunger with a stem attached to a head wherein: (i) the head is inside the container; (ii) the stem fits slidably through the cap with sufficient length to permit the head to contact the liquid in the first container; and (iii) the head moves up and down, along the longitudinal axis, through the first container as the stem slides through the cap. The head preferably contacts or engages the sidewall of the container. Further, the head has a circumference or diameter that is generally equivalent to the inner diameter or circumference of the container.

The chamber of the first container (2) is in fluid communication with the chamber of the second container (5). In one embodiment, the containers are threadably coupled. In this embodiment of the invention, water or plant homogenate is loaded into the first container (2) and flows through the second container (5).

In certain embodiments, the flow through the second container may be regulated. For example, the flow is regulated by creating positive pressure in the first container. In another example, the flow is regulated by creating negative pressure at the opening not attached to the first container. In another example, flow through the second container may be regulated by placing a liquid flow restrictor between the first and second container, or between the second and third container. Liquid flow restrictors are commonly known in the art. For example, the flow restrictor may be an aperture having a predetermined cross-sectional area. The predetermined cross-sectional area of the aperture determines the predetermined fluid flow. The flow restrictor may create a drip flow. In another example, the flow restrictor may include one or more openings. It has been contemplated that one or more pre-filters or sieves are positioned upstream of the flow restrictor in order to prevent clogging of the flow restrictor.

In yet another embodiment, the width of the second container may be equal to, or greater than the length of the second container. For example, the width may be at least 2 times, at least 3 times, at least 4 times, or at least 5 times greater than the length.

Each opening of the second container comprises a resin retention barrier. The resin retention barrier allows passage of liquid, but not passage of the particles described above. The second container contains the resin particles as described above in an aqueous solution. The resin particles are not packed. In one embodiment the aqueous solution is a buffer comprising 10-30 mM acetic acid, having a pH between 4 and 8, and 1-10 mM cysteine. The porous barrier assists in the retention of the particles described above within the chamber of the second container.

In one embodiment, the second container (5) may be provided with external threads for engaging internal threads formed in adaptor (6). The adaptor facilitates the removable coupling of the first container with the second container.

In another preferred embodiment, the chamber of the second container is in fluid communication with the chamber of a third container (8) or (10) having at least one open end. The second container is releasably coupled to the third container. In one embodiment, the second container (5) is threadably coupled with the third container (8) or (10).

In one embodiment, the second container may be provided with external threads for engaging internal threads formed in adaptor (7). The adaptor facilitates the removable coupling of the second container with third container.

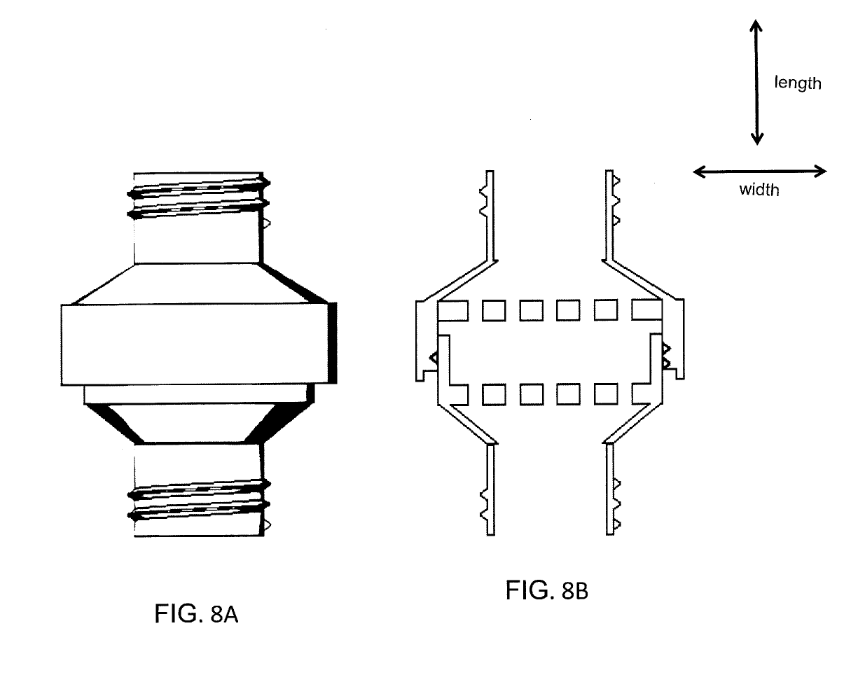

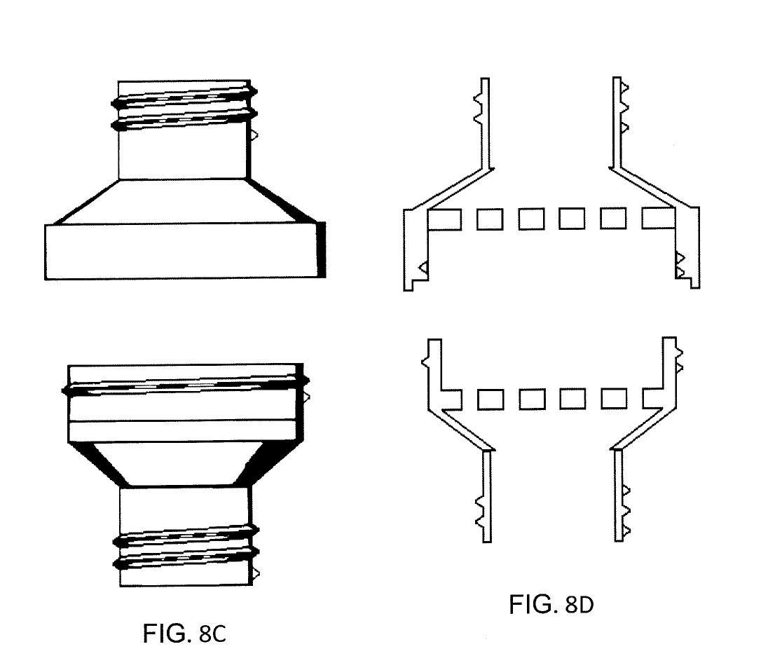

In yet another embodiment, the second container may include two components, releasably coupled to form a single second container. See FIG. 8.

In another embodiment, the third container (8) collects flow through material from passage of the aqueous suspension comprising a homogenate of plant material or water across the particles as described above, which are present in the second container (5).

In one embodiment, the third container (8) may have two openings. The first opening attaches to the second container and the second opening is opposite the first opening. Furthermore, a cap is provided for closing container upon separation from second container. Both the cap and container can be threadably connected in a conventional manner. Additionally a second cap (9) for the second opening of the third container is provided. The second cap may comprise a porous barrier as described above.

In one embodiment, the third container may have a port allowing for attachment of a pump or syringe to remove air or create a vacuum. The port may be located on a side wall of the third container (8). The removal of air or creation of a vacuum will facilitate liquid flow from the first container through the second container into the third container. The port may have a porous barrier to prevent the entry of bacteria and maintain sterility within the chamber. Furthermore, the barrier is water impermeable, but gas permeable An example of a suitable porous barrier includes Emflon and Supor R membrane, available from Pall Corporation.

In yet another embodiment, the third container (9) has one opening and collects the eluate from the particles as described above. In this embodiment, the third container may contain a buffer. For example, the third container may contain a volume equal to the eluate volume of 1M Tris HCl (pH 8.5). Alternatively, the Tris HCl may be present in solid form in an amount sufficient to bring the pH of the eluate to between 5 and 8. In a preferred embodiment, a cap is provided for closing the third container upon separation from second container (5). Both the cap and container can be threadably connected in a conventional manner.

In another embodiment, the third container is a 100, 250, or 500 ml bottle. An example of a commercially available bottle for use in this invention includes Corning® Easy Grip Round Polystyrene Storage Bottles.

In another embodiment, the third container is a vial wherein the distal end is flat, conical, or tapered. An example of a commercially available vial suitable for use in this invention includes Corning 50 mL PP Centrifuge Tubes, Conical Bottom with Plug Seal Cap (Product #430290).

In the specification, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Throughout this specification, quantities are defined by ranges, and by lower and upper boundaries of ranges. Each lower boundary can be combined with each upper boundary to define a range. The lower and upper boundaries should each be taken as independent and separate elements.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

### EXAMPLES

The present invention is illustrated in further details by the following non-limiting examples.

### Example 1

Extraction and Quantification of Bacteria Isolated from a Liquid Sample.

Swell Resin. 1.5 g of resin (A300 resin, Polysciences) was placed into sterile 50 mL conical bottom tubes. 1 mL of 1M Acetic Acid (pH 5) was added to each tube and filled to 50 mL mark with autoclaved distilled H₂O. The tube was rotated at least 2 hr at 10 rev/min at room temperature.

Preparation of inoculated samples. In a separate sterile 50 mL conical tube, the following was added: 1 mL 1M Acetic Acid (pH 5), 41.5 mL autoclaved distilled H₂O, 2.5 mL of a solution having 100 mM cysteine and 100 mM Acetic Acid (pH5), 5 μL of overnight subculture (10-1000× dilute) and 5 uL of S. cerevisiae genomic DNA (concentration adjusted to be ˜equivalent to cellular concentration for target species). The process is repeated for each sample. The solution was inverted to mix. Extract 100 uL sample for qPCR and add an equal volume of 1M Tris (pH 8.5) to correct the pH. Set aside.

Bacteria and particle/resin binding. Inoculated samples were added to the tubes containing the drained resin samples. The Mixture was placed on rotator for 2 hr. The tube was then removed from the rotator, and the resin allowed to precipitate to the bottom of the tube. Extract 100 uL sample for qPCR and add an equal volume of 1M Tris (pH 8.5) to correct the pH. Set aside.

Transfer Resin to strainer. Inoculated samples and resin were poured into a strainer cylinder. The flow through was discarded. The resin is preserved in the strainer column.

Wash. The stopcock on lower outlet of strainer column was closed. 2 mL of 20 mM Acetic Acid (pH5) was pipetted into the column. Stopcock was opened and allowed buffer to drain through the strainer. 100 uL of the flow through was collected for qPCR and add an equal volume of 1M Tris (pH 8.5) was added to correct the pH. Set aside.

Elute. Stopcock on lower outlet of strainer column was closed. 1 mL of 1M Acetic Acid (pH3) was pipetted into the strainer column. 100 uL of the flow through was collected for qPCR and an equal volume of 1M Tris (pH 8.5) was added to correct the pH. Set aside.

Serial dilutions for qPCR were prepared. 10× dilutions of the 100 uL collected (pH corrected) were prepared using samples using autoclaved distilled H₂O.

qPCR. For each reaction, 20 uL of Syto Master Mix (+Primers) and 5 uL 10× diluted template samples were used. In-House Syto9 Master Mix was prepared using: 1 U (per 25 μL volume) Roche FastStart Enzyme blend (Roche Diagnostics); 1× FastStart Buffer with MgCl2 (Roche Diagnostics); 200 μM dNTP mix; 2 μM SYTO 9 (Invitrogen; Life Technologies); 0.4 μM forward and reverse primers.

Thermocycler Protocol (Use FAM Filters for Syto9 Detection)

| | | |
|---|---|---|
| 1 cycle | 95° C. | 5 min |
| 45 cycles | 95° C. | 30 s |
| | 52° C. | 30 s |
| | — | Fluorescence |
| | | Scan |
| | 72° C. | 20 s |
| Melting | Ramp | 3 s per ° C. and |
| Curve | 60-85° C. | Fl.Scan |

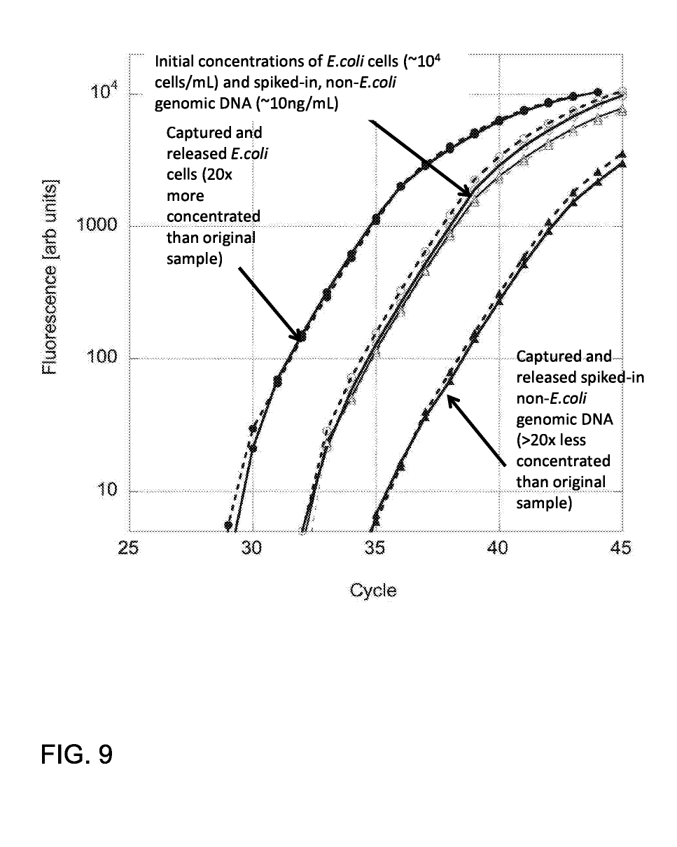

The qPCR fluorescence profile/plot is shown in FIG. 9.

Viability plating. Serial dilutions of the template samples used were prepared and plated on appropriate agar media to determine the number of CFU.

What we claim is:

1. Unbound particles comprising a resin wherein the particles:
(i) are non-magnetic;
(ii) are substantially free of cells;
(iii) are substantially free of extracellular pathogenic DNA;
(iv) are capable of forming reversible complexes with human pathogens; and
(v) have a minimum average particle diameter of 20 μm and a maximum average particle diameter of 1500 μm.

2. The particles of claim 1, wherein the resin has a bead structure.

3. The particles of claim 1, wherein the resin is substantially free of extracellular pathogenic RNA.

4. The particles of claim 1, wherein the particles have pores less than 5 μm in average diameter.

5. The particles of claim 1, wherein the particles are capable of forming reversible complexes with bacteria on the surface of the particles.

6. The particles of claim 1, wherein the resin is an anion exchange resin.

7. The particles of claim 1, wherein the resin is positively charged.

8. The particles of claim 1, wherein the particles comprise polymer substrate resins.

9. The particles of claim 1, wherein the resin is strongly basic.

10. The particles of claim 9, wherein the resin comprises quaternary ammonium groups.

11. The particles of claim 10, wherein the quaternary ammonium groups are trimethylammonium groups.

12. The particles of claim 9, wherein the resin comprises acryloyl groups.

13. The particles of claim 12, wherein the resin comprises acrylamidopropyltrimethylammonium groups.

14. The particles of claim 13, wherein the resin comprises acrylamidopropyltrimethylammonium chloride.

15. The particles of claim 1, wherein the particles comprise polystyrene with divinyl benzene cross linked matrices.

16. The particles of claim 1, wherein the particles comprise weakly basic resins.

17. The particles of claim 16, wherein the weakly basic resins comprise primary, secondary, or tertiary amino groups.

18. The particles of claim 17, wherein the resin comprises polyethylene amine.

19. The particles of claim 15, wherein the particles comprise polystyrene cross-linked with divinylbenzene.

20. The particles of claim 1, wherein the resin is a cation exchange resin.

21. The particles of claim 1, wherein the resin is negatively charged

22. The particles of claim 1, wherein the resin is strongly acidic.

23. The particles of claim 22, wherein the resin comprises sulfonic acid groups or phosphonic acid groups.

24. The particles of claim 1, wherein the particles comprise weakly acidic resins.

25. The particles of claim 24, wherein the resin comprises acrylic acid, or carboxylic acid.

26. The particles of claim 25, wherein the resin comprises methacrylic acid.

27. The particles of claim 1, wherein the particles are selected from the group consisting of: Diaion™ Acrylic Gel, Diaion™ Highly Porous, and Polyscience A300.

28. The particles of claim 1, wherein the particles have a minimum average particle diameter of 150 μm.

29. The particles of claim 1, wherein the particles have a minimum average particle diameter of 300 μm.

30. The particles of claim 1, wherein the particles have a maximum average particle diameter of 1200 μm.

31. The particles of claim 1, wherein the particles have a maximum average particle diameter of 1500 μm.

32. The particles of claim 1, wherein the particles are monodisperse.

33. The particles of claim 1, wherein the particles do not comprise a coating.

34. The particles of claim 1, wherein the particles do not comprise an antibody.

35. The particles of claim 1, wherein the particles are not packed in a column.

36. The particles of claim 1, wherein the particles are not bound in a matrix.

37. The particles of claim 1, wherein the particles are not bound in a membrane or a film.

38. The particles of claim 1, wherein the particles are suspended in water.

39. The particles of claim 1, wherein the particles are in an aqueous suspension comprising a homogenate of plant material or water, and human pathogens.

40. A reversible complex comprising a human pathogen and a particle according to claim 1.

41. The complex of claim 40, wherein the human pathogen is a gram negative bacterial cell.

42. The complex of claim 40, wherein the human pathogen is a gram positive bacterial cell.

43. The complex of claim 40, wherein the human pathogen is selected from the group of genera consisting of Escherichia, Salmonella, Listeria, Shigella, Vibrio, Clostridium and Campylobacter.

44. The complex of claim 40, wherein the pathogen is from the genus Escherichia.

45. The complex of claim 40, wherein the pathogen is from the species E. coli.

46. The complex of claim 45, wherein the E. coli bacteria are selected from the group of entrovirulent E. coli consisting of enterohaemorragic (EHEC), enterotoxigenic (ETEC), enteroinvasive (EIEC), and Shiga-like toxin producing (STEC) cells.

47. The complex of claim 40, wherein the human pathogen is selected from the genus Salmonella.

48. The complex of claim 47, wherein the Salmonella is selected from the group of species consisting of S. enterica and S. typhimurium.

49. The complex of claim 47, wherein the Salmonella is selected from the group of subspecies consisting of enterica, salamae, arizonae, diarizonae, houtenae, and indica.

50. The complex of claim 40, wherein the pathogen is from the genus Listeria.

51. The complex of claim 50, wherein the pathogen is from the species L. monocytogenes.

52. The complex of claim 40, wherein the pathogen is selected from the genus Cronobactor.

53. The complex of claim 52, wherein the pathogen is from the species C. sakazakii.

54. The complex of claim 40, wherein the pathogen is selected from the genus Campylobacter.

55. The complex of claim 54, wherein the pathogen is selected from the group of species consisting of C. jejuni, C. coli, and C. jari.

56. The complex of claim 40, wherein the pathogen is selected from the genus Shigella.

57. The complex of claim 56, wherein the pathogen is selected from the group of species consisting of S. dysenteriae, S. flexneri, S. boydii, and S. sonnei.

58. The complex of claim 40, wherein the pathogen is selected from the genus Vibrio.

59. The complex of claim 58, wherein the pathogen is selected from the group of species consisting of V. parahaemolyticus, V. cholerae, and V. vulnificus.

60. The complex of claim 40, wherein the pathogen is selected from the genus Clostridium.

61. The complex of claim 60, wherein the pathogen is from the species C. botulinum.

62. The complex of claim 40, wherein the pathogen is a virus.

63. The complex of claim 62, wherein the virus is selected from the group consisting of hepatitis C virus and norovirus.

64. The complex of claim 40, wherein the pathogen is a fungus.

65. The complex of claim 64 wherein the fungus is from the genus Aspergillus.

66. The complex of claim 65, wherein the fungus is from the group of species consisting of A. fumigatus and A. flavus.

67. A method for isolating a human pathogen from liquid, the method comprising:
(a) preparing an aqueous suspension by contacting:
(i) a liquid comprising human pathogens;
(ii) particles comprising a resin according to claim 1; and
(iii) optionally a first solution
(b) for a time sufficient to form a complex between the human pathogens and the resin particles;
(c) separating the liquid from the complex; and
(d) separating the human pathogen from the complex with a second solution thereby obtaining an aqueous mixture comprising the human pathogen sufficiently free of other sources of DNA to permit identification of the human pathogen,
wherein:
if the human pathogen is a cell, the cell remains intact after step (c);
the first solution and the second solution are sterile, and substantially DNA-free;
the second solution is compatible with an immunoassay or a qPCR assay; and
the pH of the first solution is different from the pH of the second solution.

68. The method of claim 67, wherein the liquid is water or a plant homogenate.

69. The method of claim 67, wherein a majority of the cells separated in step (c) are viable.

70. The method of claim 67, wherein the resin is strongly basic.

71. The method of claim 70, wherein the second solution has a minimum pH of 3 and a maximum pH of about 6.

72. The method of claim 70, wherein the first solution has a minimum pH of about 5 and a maximum pH of about 9.

73. The method of claim 67, wherein the resin is weakly basic.

74. The method of claim 73, wherein the second solution has a minimum pH of 3 and a maximum pH of about 6.

75. The method of claim 73, wherein the first solution has a minimum pH of about 5 and a maximum pH of about 9.

76. The method of claim 73, wherein the second solution is tris-ethylenediamene tetraacetic acid.

77. The method of claim 67, wherein the resin is strongly acidic.

78. The method of claim 77, wherein the second solution has a minimum pH of about 3 and a maximum pH of about 6.

79. The method of claim 77, wherein the first solution has a minimum pH of about 7 and a maximum pH of about 9.

80. The method of claim 67, wherein the resin is weakly acidic.

81. The method of claim 80, wherein the second solution has a minimum pH of about 3 and a maximum pH of about 6.

82. The method of claim 80, wherein the first solution has a minimum pH of about 7 and a maximum pH of about 9.

83. A method for determining the number of human pathogens in a liquid, the method comprising:
(a) contacting:
(i) a liquid comprising pathogens and other sources of DNA; and
(ii) sterilized non-magnetic particles comprising a resin according to claim 1;
in first solution for a time sufficient to form a complex between the pathogens and the particles;
(b) separating the liquid and the water from the complex;
(c) eluting the pathogens from the complex with a second solution thereby obtaining an eluate comprising the pathogens sufficiently free of the other sources of DNA to permit determining the number of the pathogens; and
(d) determining the number of pathogens in the eluate.

84. The method of claim 83, wherein the liquid is water.

85. The method of claim 83, wherein the liquid is a plant homogenate.

86. The method of claim 83, wherein the resin is strongly basic.

87. The method of claim 86, wherein the second solution has a minimum pH of about 3 and a maximum pH of about 6.

88. The method of claim 86, wherein the first solution has a minimum pH of about 5 and a maximum pH of about 9.

89. The method of claim 83, wherein the second solution is 0.1M acetic acid.

90. The method of claim 83, wherein the resin is weakly basic.

91. The method of claim 90, wherein the second solution has a minimum pH of about 3 and a maximum pH of about 6.

92. The method of claim 90, wherein the first solution has a minimum pH of about 5 and a maximum pH of about 9.

93. The method of claim 83, wherein the second solution is tris-ethylenediamene tetraacetic acid.

94. The method of claim 83, wherein the resin is strongly acidic.

95. The method of claim 94, wherein the second solution has a minimum pH of about 3 and a maximum pH of about 6.

96. The method of claim 94, wherein the first solution has a minimum pH of about 7 and a maximum pH of about 9.

97. The method of claim 83, wherein the resin is weakly acidic.

98. The method of claim 97, wherein the second solution has a minimum pH of about 3 and a maximum pH of about 6.

99. The method of claim 97, wherein the first solution has a minimum pH of about 7 and a maximum pH of about 9.

100. The method of claim 83, wherein the number of pathogens in the eluate is determined by qPCR.

101. The method of claim 83, wherein the number of pathogens in the eluate is determined by a CFU plating assay.

102. A method for determining whether the number of human pathogens in a liquid exceeds a threshold level of pathogenicity, the method comprising:
(a) contacting:
(i) a liquid comprising human pathogens and other sources of DNA; and
(ii) sterilized non-magnetic particles comprising a resin according to claim 1;
in sterile water for a time sufficient to form a complex between the pathogens and the particles;
(b) separating the homogenate and the water from the complex; and
(c) eluting the pathogens from the complex with a second solution thereby obtaining an eluate comprising the pathogens sufficiently free of the other sources of DNA to permit determining whether the number of pathogens in the eluate exceeds a threshold level.
(d) establishing the threshold level of pathogenicity; and
(e) determining whether the number of pathogens in the eluate exceeds the threshold level of pathogenicity.

103. The method of claim 102, wherein the liquid is water.

104. The method of claim 102, wherein the liquid is a plant homogenate.

105. The method of claim 102, wherein the resin is strongly basic.

106. The method of claim 105, wherein the second solution has a minimum pH of 3 and a maximum pH of about 6.

107. The method of claim 105, wherein the first solution has a minimum pH of about 5 and a maximum pH of about 9.

108. The method of claim 105, wherein the second solution is 0.1M acetic acid.

109. The method of claim 102, wherein the resin is weakly basic.

110. The method of claim 109, wherein the second solution has a minimum pH of about 3 and a maximum pH of about 6.

111. The method of claim 109, wherein the first solution has a minimum pH of about 5 and a maximum pH of about 9.

112. The method of claim 109, wherein the second solution is tris-ethylenediamene tetraacetic acid.

113. The method of claim 102, wherein the resin is strongly acidic.

114. The method of claim 113, wherein the second solution has a minimum pH of about 3 and a maximum pH of about 6.

115. The method of claim 113, wherein the first solution has a minimum pH of about 7 and a maximum pH of about 9.

116. The method of claim 102, wherein the resin is weakly acidic.

117. The method of claim 116, wherein the second solution has a minimum pH of about 3 and a maximum pH of about 6.

118. The method of claim 116, wherein the first solution has a minimum pH of about 7 and a maximum pH of about 9.

119. The method of claim 102, wherein the number of pathogens in the eluate is determined by qPCR.

120. The method of claim 102, wherein the number of pathogens in the eluate is determined by a CFU plating assay.

121. A system for isolating human pathogens from a liquid comprising a suspension and a device:
an aqueous suspension of resin particles comprising:
(i) particles comprising a resin according to claim 1, and
(ii) optionally a buffer;
the device comprising:
a first container comprising an internal main chamber, inlet opening, and an outlet opening;
a second container comprising an internal main chamber, inlet opening removably coupled to said first container, and outlet opening;
wherein said internal main chamber of the second container comprises said resin;
wherein the inlet opening and outlet opening of the second container comprises a porous barrier that allows passage of liquid, but does not allow passage of said resin;
wherein first container is in fluid communication with the second container.

122. A system as defined in claim 121, wherein the liquid comprises a homogenate of plant material comprising human pathogens.

123. A system as defined in claim 121, wherein the liquid comprises water containing human pathogens.

124. A system as defined in claim 123, wherein the water is used in agriculture.

125. A system as defined in claim 123, wherein the water is used to wash produce.

126. A device as defined in claim 121, further comprising a cap removably attached to said first container for permitting access to said internal main chamber.

127. A device as defined in claim 121, wherein the first container further comprises an inlet port permitting access to said internal main chamber of the first container, wherein the inlet port comprises a porous filter.

128. A device as defined in claim 121, further comprising a third container comprising an internal main chamber having an inlet opening, wherein the third container is removably coupled to said second container by the inlet opening; wherein said chamber of second container is in fluid communication with the chamber of said third container.

129. A device as defined in claim 128, wherein said third container comprises a port for attachment of a syringe or a vacuum pump.

130. A device as defined in claim 121, wherein said first container is threadably coupled to said second container.

131. A device as defined in claim 128, wherein said second container is threadably coupled to said third container.

132. A third container as defined in claim 131, further comprising a cap capable of removable attachment to the opening of said third container.

133. A device as defined in claim 121, wherein the device is sterile.
